(19) United States
(12) Patent Application Publication
(10) Pub. No.: US 2012/0122221 A1
(43) Pub. Date: May 17, 2012

(54) CULTURE MEDIUM AND HYDROPHILIC COMPOSITE THEREOF

(75) Inventors: CHEN FENG, Beijing (CN); LI FAN, Beijing (CN); WEN-MEI ZHAO, Beijing (CN)

(73) Assignees: HON HAI PRECISION INDUSTRY CO., LTD., Tu-Cheng (TW); TSINGHUA UNIVERSITY, Beijing (CN)

(21) Appl. No.: 13/294,375

(22) Filed: Nov. 11, 2011

(30) Foreign Application Priority Data
Nov. 12, 2010 (CN) 201010541533.2

(51) Int. Cl. C12N 5/02; C07K 14/00; B82Y 5/00

(52) U.S. Cl. 435/402; 530/350; 977/746; 977/923

(57) ABSTRACT

A hydrophilic composite includes a carbon nanotube structure and a protein layer. The carbon nanotube structure has at least one carbon nanotube film. The protein layer covers one surface of the carbon nanotube structure, and is coupled to the at least one carbon nanotube film. The carbon nanotube structure is disposed on a substrate.

This application claims all benefits accruing under 35 U.S.C. §119 from China Patent Application No. 201010541533.2, filed on Nov. 12, 2010 in the China Intellectual Property Office, disclosure of which is incorporated herein by reference.

### CROSS-REFERENCES TO RELATED APPLICATIONS

This application is related to commonly-assigned applications entitled, “METHOD FOR FORMING HYDROPHILIC COMPOSITE,” filed ______ (Atty. Docket No. US35619).

### BACKGROUND

1. Technical Field

The present disclosure relates to a culture medium and a hydrophilic composite including carbon nanotubes thereof.

2. Description of Related Art

Since 1991, carbon nanotube is found by Sumino Iijima. The carbon nanotube has become into a promising material applied in a variety of applications, such as electrics, optics, nanotechnology and other fields of material science.

Although carbon nanotubes have proven to be a useful material, their hydrophobic nature which tend to aggregate in the solvent may be problematic. Generally, the chemical treatment is used to change the carbon nanotubes from hydrophobic to hydrophilic, so that carbon nanotubes are able to disperse evenly in the solution. This can be done by introducing certain molecules or functional groups, such as phenolic (OH) or carboxyl (COOH) group, chemically onto the surface of the carbon nanotube is a general way to make carbon nanotubes more easily dispersible in liquid and not to significantly change the desired properties of carbon nanotubes. The above described process is called functionalization in which an acid solution is used and through acid-oxidation reaction to modify the carbon nanotubes.

However, the acid solution, such as nitric acid, used in functionalization is difficult to be removed from the functionalized carbon nanotubes. What is needed, therefore, is to provide a hydrophilic composite, to overcome the above-described shortcomings.

### BRIEF DESCRIPTION OF THE DRAWINGS

Many aspects of the disclosure can be better understood with reference to the drawings. The components in the drawings are not necessarily drawn to scale, the emphasis instead being placed upon clearly illustrating the principles of the present disclosure. Moreover, in the drawings, like reference numerals designate corresponding parts throughout the several views.

FIG. 1 shows a transmission electron microscope (TEM) image of one embodiment of a hydrophilic composite.

FIG. 2 is a schematic view of the hydrophilic composite shown in FIG. 1.

FIG. 3 shows a scanning electron microscope (SEM) image of the hydrophilic composite shown in FIG. 1.

FIG. 4 is a cross-sectional view of the hydrophilic composite shown in FIG. 1.

FIG. 5 shows a SEM image of one embodiment of a carbon nanotube film.

FIG. 6 shows a schematic view of a process for forming the hydrophilic composite shown in FIG. 1.

FIG. 7 shows a TEM image of one embodiment of stacked carbon nanotube films.

FIG. 8 is a schematic view of another embodiment of a hydrophilic composite.

FIG. 9 is a cross-sectional view of the hydrophilic composite shown in FIG. 8.

FIG. 10 shows a schematic view of a process for forming the hydrophilic composite shown in FIG. 8.

### DETAILED DESCRIPTION

The disclosure is illustrated by way of example and not by way of limitation in the figures of the accompanying drawings in which like references indicate similar elements. It should be noted that references to “an” or “one” embodiment in this disclosure are not necessarily to the same embodiment, and such references mean at least one.

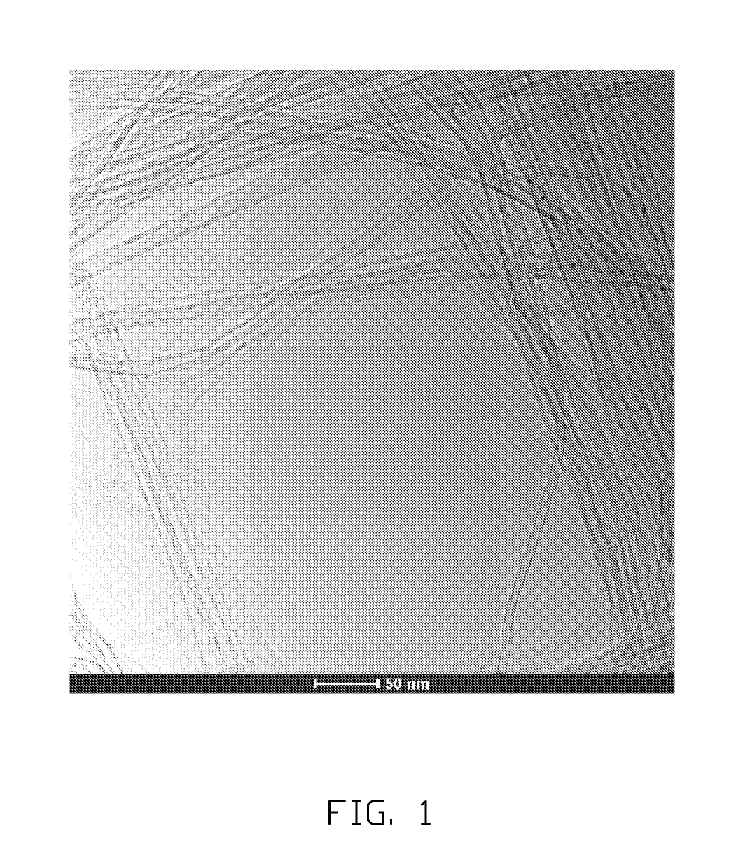

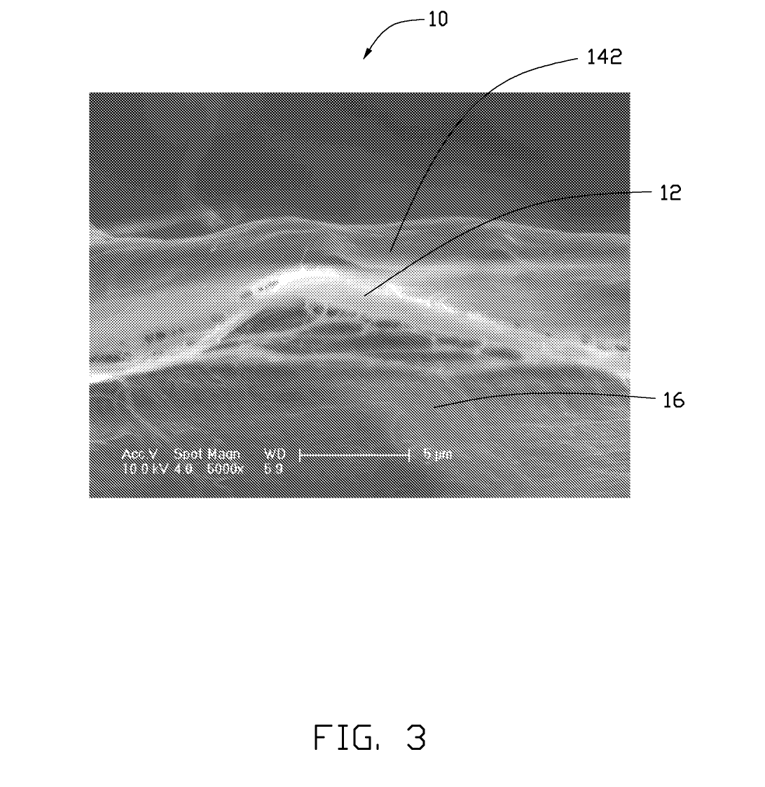

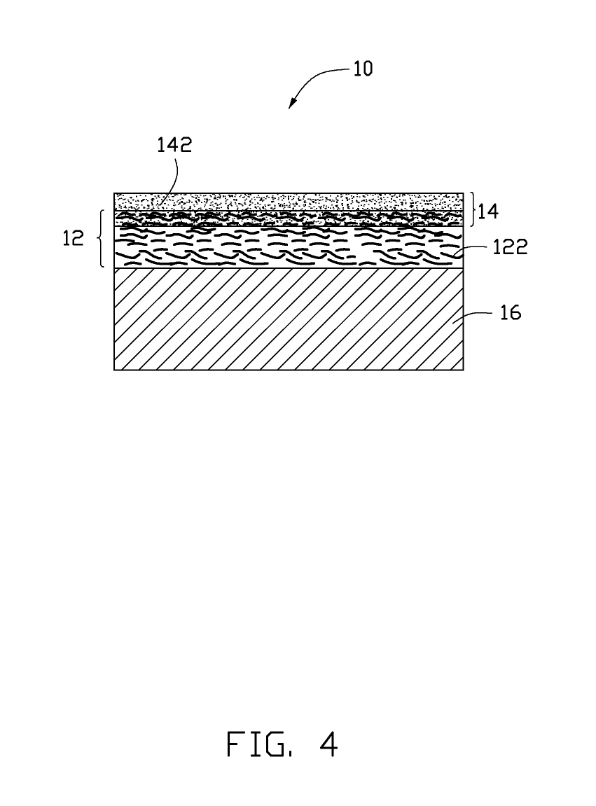

Referring to FIGS. 1-4, one embodiment of a hydrophilic composite 10 includes a carbon nanotube structure 12, a protein layer 14, and a substrate 16. The carbon nanotube structure 12 is disposed on the substrate 16. In one embodiment, a surface of the carbon nanotube structure 12 can be flat or curved.

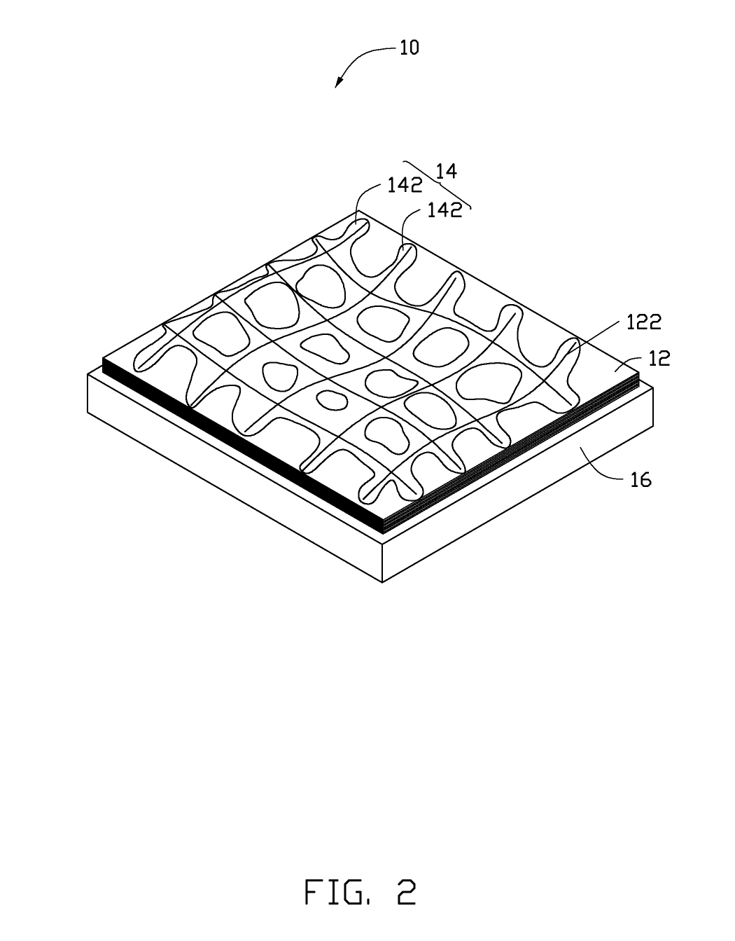

The carbon nanotube structure 12 includes a number of carbon nanotube films, which are stacked together. Each of the carbon nanotube films includes a number of carbon nanotubes 122. For example, the carbon nanotube structure 12 is formed by 10 layers of carbon nanotube films (as shown in FIG. 2).

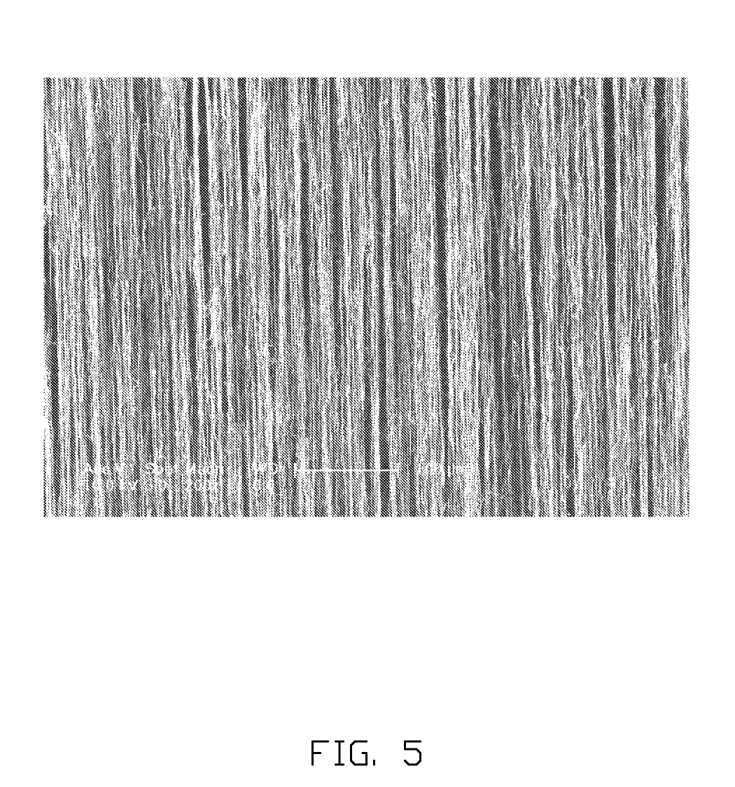

Referring to FIG. 5, each of the carbon nanotube films is formed by drawing portion of carbon nanotubes in a carbon nanotube array, which is formed by a number of super-aligned carbon nanotubes arranged on a substrate. Each carbon nanotube film includes a number of successive and oriented carbon nanotubes joined end to end by van der Waals attractive force. That is, the carbon nanotubes in each of the carbon nanotube films are primarily orientated in one direction. In addition, the carbon nanotubes are substantially parallel to the surface of the carbon nanotube structure 12. When the carbon nanotube films are stacked sequentially, the orientations of the carbon nanotubes in two adjacent carbon nanotube films can be intersected at an angle. Thus, the stacked carbon nanotube films can be formed a porous carbon nanotube structure. In one embodiment, the angle is in a range from about 0 degree to about 90 degrees.

For example, as shown in FIG. 2, one of orientations of carbon nanotubes is substantially perpendicular to the other of orientations of carbon nanotubes. Consequently, the carbon nanotube structure 12 forms a mesh-like structure. The mesh-like structure defines a number of holes having an average diameter which is in a range from about 1 nanometer (nm) to about 10 micrometers (μm). It is understood that the dimensions of the hole is corresponding to the layer number of the carbon nanotube films. That is, the more layers of carbon nanotube films are chosen to form the carbon nanotube structure 12, the smaller holes are defined.

The carbon nanotubes in two adjacent carbon nanotube films are combined and attracted by van der Waals attractive force, forming a free-standing structure. That is, the free-standing carbon nanotube structure is able to stand in a particular shape without any supporter. It is noteworthy that the number of carbon nanotubes films and the angle made by adjacent carbon nanotube films are arbitrary and set according to practical requirements.

Alternatively, the carbon nanotube structure 12 can be formed by a number of carbon nanotube wires. Thus, one portion of the carbon nanotube wires is arranged parallel to each other and extends along a first direction. In addition, the other portion of the carbon nanotube wires is arranged parallel to each other and extends along a second direction. The first direction and the second direction can be substantially perpendicular to each other. In one embodiment, the carbon nanotube wire can be classified as untwisted carbon nanotube wire and twisted carbon nanotube wire. The untwisted carbon nanotube wire is made by treating an organic solvent to the carbon nanotude film described above. In such case, the carbon nanotubes of the untwisted carbon nanotube wire are parallel to the axis of the carbon nanotube wire. In one embodiment, the organic solvent can be ethanol, methanol, acetone, dichloroethane, or chloroform. The diameter of the untwisted carbon nanotube wire is in a range from about 0.5 nm to about 1 millimeter.

Furthermore, the carbon nanotube wire can be formed by twisting the carbon nanotube film to form the twisted carbon nanotube wire. Specifically, twisted carbon nanotube wire is formed by turning two opposite ends of the carbon nanotube film to opposite directions. In one embodiment, the carbon nanotubes of the carbon nanotube wire are aligned around the axis of the carbon nanotube spirally.

The substrate 16 can be a hydrophobic substrate capable of absorbing the carbon nanotube structure 12. That is, the carbon nanotube structure 12 can adhere to the substrate 16 without any adhesive. In addition, the substrate 16 can be a hard substrate or a flexible substrate according to the practical needs. For example, the substrate 12 can be made of hard material such as ceramic, glass, or quartz as a hard substrate. Alternatively, the substrate 12 can be made of flexible material such as silicon dioxide. Thus, the hydrophilic composite 10 is capable to be bent and employed to structures with different shapes. In one embodiment, the substrate 12 is made of silicon dioxide. The hydrophilic composite 10 can be employed in biotechnology field, e.g. serving as a cell culture medium.

The protein layer 14 including soluble proteins 142 covers at least a portion of one surface of the carbon nanotube structure 12. To form the protein layer 14, the carbon nanotube structure 12 is immersed in a protein solution, including soluble proteins 142 selected from a group consisting of bovine serum, porcine serum, equine serum, goat serum, and combination thereof. The soluble proteins 142 of the protein layer 14 can interact with the carbon nanotubes 122 of the carbon structure 12. Specifically, the soluble proteins 142 can be absorbed onto the surface of the carbon nanotube structure 12 and linked to the carbon nanotubes 122.

In one embodiment, the protein layer 14 can be a continuous layer with a specific thickness on the carbon nanotube structure 12. In addition, the soluble proteins 142 of the protein layer 14 can also penetrate into the carbon nanotube structure 12 through holes formed by mesh-like carbon nanotube structure. Thus, the protein layer 14 not only covers the carbon nanotube structure 12 but also penetrates the portion of the surface of carbon nanotube structure 12. Consequently, the soluble proteins 142 may interact with the internal carbon nanotubes of the carbon nanotube structure 12. In one embodiment, the soluble proteins 142 are probably serving as hydrophilic groups in the carbon nanotube structure 12. The thickness of the protein layer 14 is in a range from about 1 nm to about 200 nm. Preferably, the protein layer 14 is with a thickness in a range from about 1 nm to about 100 nm.

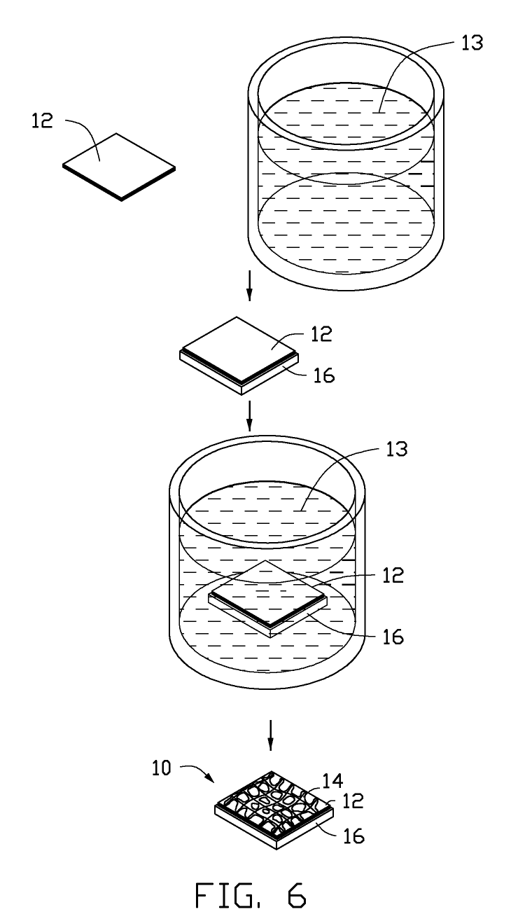

Referring to FIG. 6, a method forming a hydrophilic composite 10 includes the steps of:

S110, providing a substrate 16 and a carbon nanotube structure 12 including a number of carbon nanotubes;

S120, disposing the carbon nanotube structure 12 on the substrate 16;

S130, providing a protein solution 13; and

S140, immersing the substrate 16 with the carbon nanotube structure 12 in the protein solution 13 to form a protein layer 14 on the carbon nanotube structure 12, and soluble proteins of the protein solution 13 bind to the carbon nanotubes of the carbon nanotube structure 12.

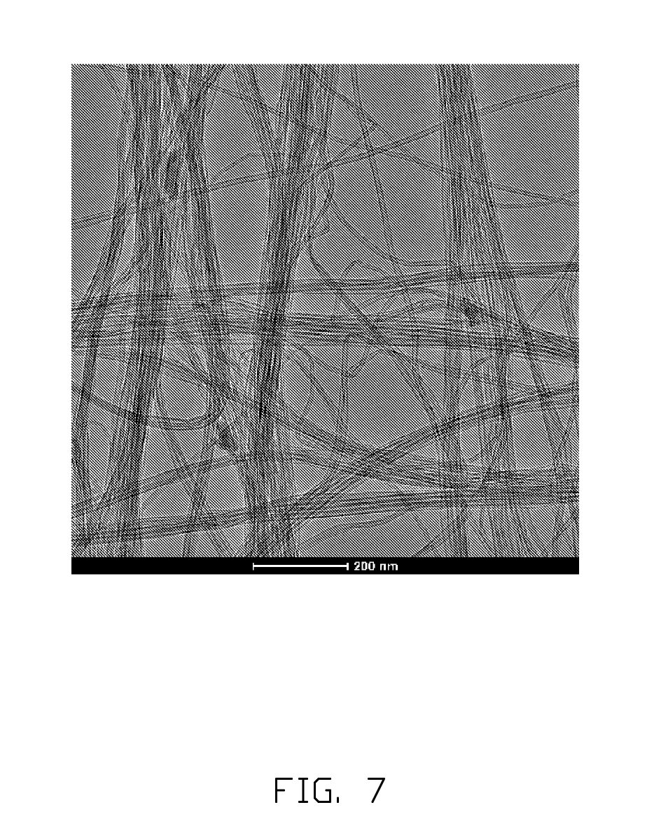

In the step 110, the carbon nanotube structure 12 is formed by 10 layers of carbon nanotube films as shown in FIG. 7. Each of the carbon nanotube films is formed by drawing portion of carbon nanotubes in a carbon nanotube array, which is formed by a number of super-aligned carbon nanotubes arranged on a substrate. Each carbon nanotube film includes a number of successive and oriented carbon nanotubes joined end to end by van der Waals attractive force. When the carbon nanotube films are stacked sequentially, the orientations of the carbon nanotubes in two adjacent carbon nanotube films can be perpendicularly intersected.

One embodiment of a method for making a carbon nanotube film of the carbon nanotube structure 12 includes the following steps:

S111, providing a carbon nanotube array on a substrate; and

S112, pulling a drawn carbon nanotube film out from the carbon nanotube array.

In the step 111, the carbon nanotube array can be a super-aligned array of carbon nanotubes. However, any carbon nanotube array from which a film can be drawn may be used. The carbon nanotube array can be formed by the steps of:

(a1), providing a substantially flat and smooth substrate;

(b1), forming a catalyst layer on the substrate;

(c1), annealing the substrate with the catalyst layer thereon in air at a temperature in a range from about 700° C. to about 900° C. for about 30 minutes to about 90 minutes;

(d1), heating the substrate with the catalyst layer thereon at a temperature in a range from about 500° C. to about 740° C. in a furnace with a protective/reducing gas therein; and

(e1), supplying a carbon source gas to the furnace for about 5 minutes to about 30 minutes, and growing a carbon nanotube array of carbon nanotubes on the substrate.

In the step (a1), the substrate can be a P-type silicon wafer, an N-type silicon wafer, or a silicon wafer with a film of silicon dioxide thereon. In one embodiment, a four inch P-type silicon wafer is used as the substrate. In the step (b1), the catalyst can be made of iron (Fe), cobalt (Co), nickel (Ni), or any combination thereof.

In the step (d1), the protective/reducing gas can be made of at least one of nitrogen (N₂), ammonia (NH₃), and a noble gas. In the step (e1), the carbon source gas can be a hydrocarbon gas, such as ethyne (C₂H₂), ethylene (C₂H₄), methane (CH₄), ethane (C₂H₆), or any combination thereof. In one embodiment, the protective/reducing gas is argon, and the carbon source gas is ethyne.

In one embodiment, the carbon nanotubes in the carbon nanotube array have a height of about 100 μm. The carbon nanotube array formed under the above conditions is essentially free of impurities, such as carbonaceous or residual catalyst particles. The carbon nanotubes in the carbon nanotube array are closely packed together by the van der Waals force.

In the step S112, the drawn carbon nanotube film can be pulled out of the carbon nanotube array by the steps of: (a2), contacting the carbon nanotube array with an adhesive bar; and (b2), moving the adhesive bar away from the carbon nanotube array.

In the step (a2), the adhesive bar can include a body with a side surface covered by an adhesive layer. The side surface of the body can be made of a material having a great attractive force to the carbon nanotubes. Therefore, the side surface of the body can be used as a contacting surface to contact a number of carbon nanotubes of the carbon nanotube array, and the carbon nanotubes can be firmly adhered to the side surface of the adhesive bar. The adhesive bar can be fixed to a stretching device via a fixing device.

In the step (b2), if the adhesive bar is driven to move away from the carbon nanotube array, a number of carbon nanotube segments can be pulled out from the carbon nanotube array end-to-end to form the drawn carbon nanotube film due to the van der Waals force between adjacent carbon nanotube segments. During the pulling process, an angle between a direction of pulling the drawn carbon nanotube film and the longitudinal direction of the carbon nanotube array can be in a range from about 30 degrees to about 90 degrees.

The carbon nanotube film of the carbon nanotube structure 12 also can be formed by entangled carbon nanotubes. Alternatively, the carbon nanotube film of the carbon nanotube structure 12 can be formed of a plurality of carbon nanotubes arranged isotropically.

In the step 120, the carbon nanotube structure 12 is inherently adhesive due to carbon nanotubes with high specific surface. Thus, the carbon nanotube structure 12 is adhered to the substrate 16 easily once the carbon nanotube structure 12 being disposed on the substrate 16.

In the step 130, the concentration of the protein solution 13 is in a range from about 0.01% (v/v %) to about 50% (v/v %). Preferably, the concentration of the protein solution 13 is in a range from about 0.1% (v/v %) to about 10% (v/v %). The solutes dissolved in the solution can be chosen from bovine serum, porcine serum, equine serum, goat serum or combination thereof. In one embodiment, the protein solution 13 is bovine serum solution with the concentration of about 1% (v/v %).

In the step 140, the substrate 16 with the carbon nanotube structure 12 is soaked into the protein solution 13 for a while. The period for immersing the substrate 16 with the carbon nanotube structure 12 is dependent on the practice needs. For example, the immersed period can be in a range from about 1 hour to about 48 hours. It is understood that the thickness of the protein layer 14 is relative to the immersing period. In one embodiment, the substrate 16 with the carbon nanotube structure 12 including 10 layers of carbon nanotube films is immersed in the bovine serum solution for 2 hours.

The carbon nanotube structure 12 serves as a scaffold where the proteins in protein solution 13 accumulated. It is understood that the hydrophilic composite 10 is shaped up by the carbon nanotube structure 12. In addition, the hydrophilic composite 10 can be treated by sterilization in order to be applied in biomedical field or be suitable to store long term. For example, the hydrophilic composite 10 is sterilized by treating at high temperature or freezing. In one embodiment, the hydrophilic composite 10 is treated at 120° C. to be sterilized.

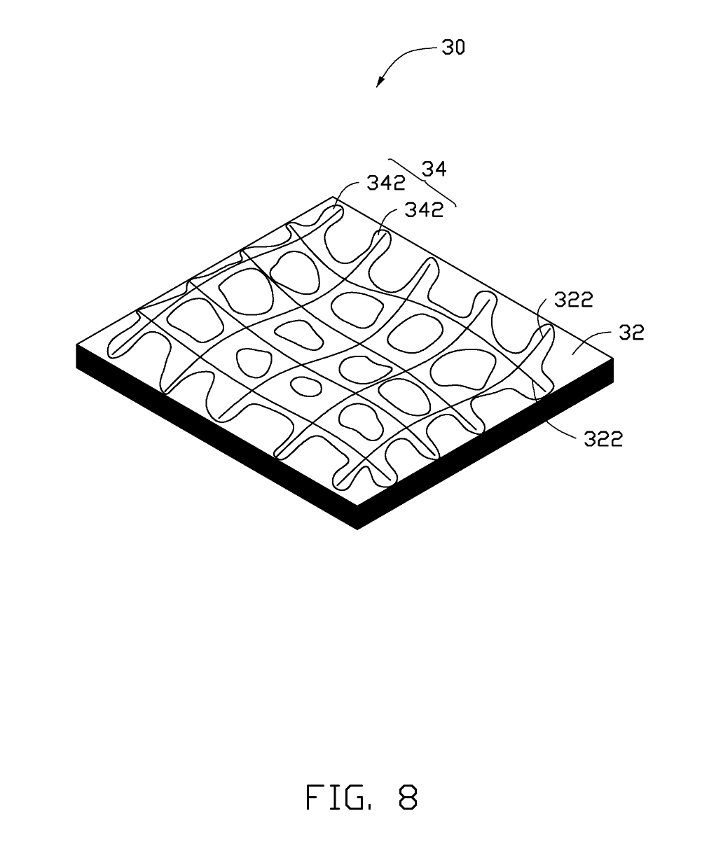

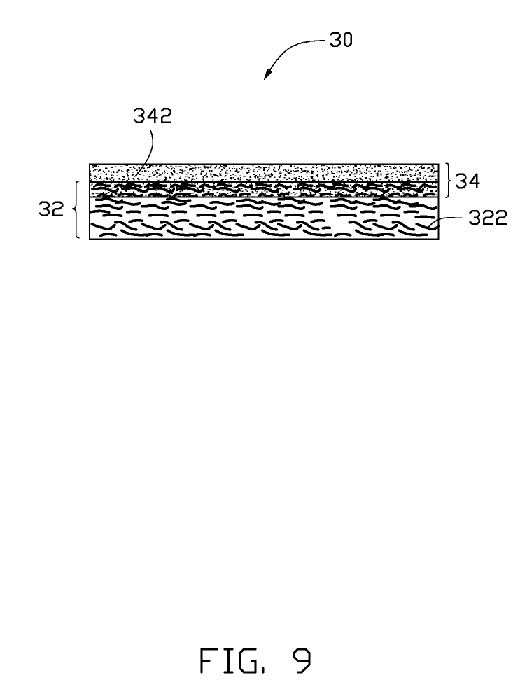

Referring to FIG. 8 and FIG. 9, one embodiment of a hydrophilic composite 30 includes a carbon nanotube structure 32 and a protein layer 34. In one embodiment, a surface of the carbon nanotube structure 32 can be flat or curved. The carbon nanotube structure 32 includes a number of carbon nanotube films, which are stacked together. Each of the carbon nanotube films includes a number of carbon nanotubes 322. For example, the carbon nanotube structure 32 is formed by 30 layers of carbon nanotube films.

The carbon nanotubes are substantially parallel to the surface of the carbon nanotube structure 32. When the carbon nanotube films are stacked sequentially, the orientations of the carbon nanotubes in two adjacent carbon nanotube films can be intersected at an angle. Thus, the stacked carbon nanotube films can form a porous carbon nanotube structure. In one embodiment, the angle is in a range from about 0 degree to about 90 degrees.

For example, one orientation of the carbon nanotubes is substantially perpendicular to the another orientation of the carbon nanotubes. Consequently, the carbon nanotube structure 32 is formed to be a mesh-like structure. Thus, the mesh-like structure defines a number of holes having an average diameter which is in a range from about 1 nm to about 10 μm. It is understood that the dimensions of the hole is corresponding to the layer number of the carbon nanotube films. That is, the more layers of carbon nanotube films are chosen to form the carbon nanotube structure 32, the smaller holes are defined.

The carbon nanotubes in two adjacent carbon nanotube films are combined and attracted by van der Waals attractive force, forming a free-standing structure. That is, the free-standing carbon nanotube structure is able to stand in a particular shape without any support. It is noteworthy that the number of carbon nanotubes films and the angle made by adjacent carbon nanotube films are arbitrary and set according to practical requirements.

The protein layer 34 including soluble proteins 342 covers at least a portion of one surface of the carbon nanotube structure 32. The protein layer 34 can be added by immersing the carbon nanotube structure 32 in a protein solution, including soluble proteins 342 selected from a group consisting of bovine serum, porcine serum, equine serum, goat serum, and combination thereof. The soluble proteins 342 of the protein layer 34 can interact with the carbon nanotubes 322 of the carbon structure 32. Specifically, the soluble proteins 342 can be absorbed onto the surface of the carbon nanotube structure 32 and linked to the carbon nanotubes 322.

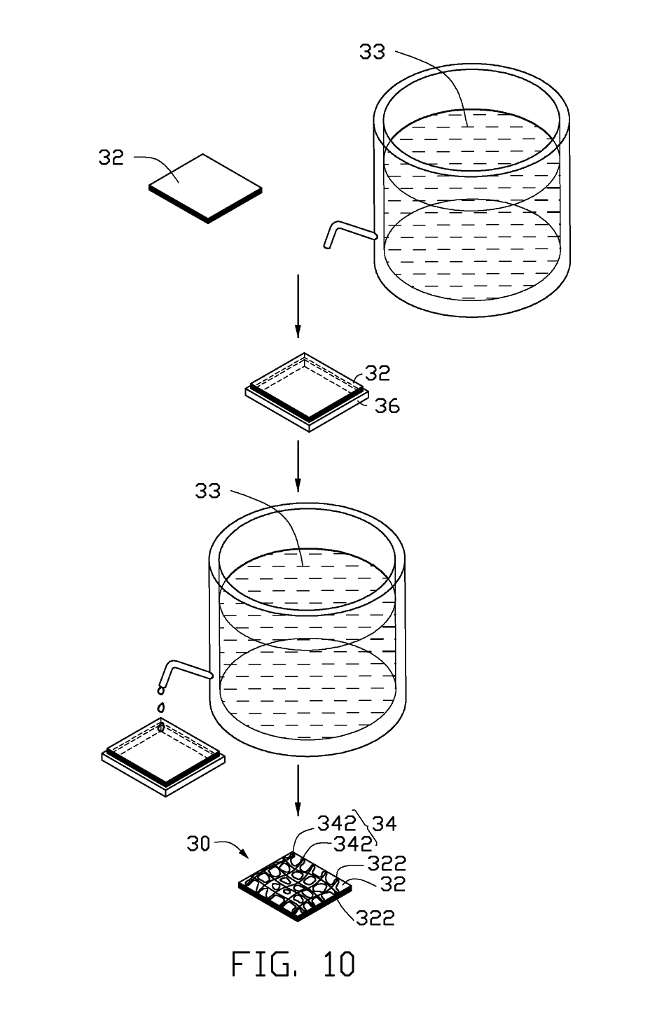

Referring to FIG. 10, a method forming a hydrophilic composite 30 includes the steps of:

S210, providing a carbon nanotube structure 32 including a number of carbon nanotubes;

S220, providing a protein solution 33; and

S230, laying the protein solution 33 on the carbon nanotube structure 32 such that a protein layer 34 is formed on the carbon nanotube structure 32, and soluble proteins of the protein layer 34 bind to the carbon nanotubes of the carbon nanotube structure 32.

In the step 210, the carbon nanotube structure 32 is formed by 30 layers of carbon nanotube films. Each of the carbon nanotube films is formed by drawing portion of carbon nanotubes in a carbon nanotube array, which is formed by a number of super-aligned carbon nanotubes arranged on a substrate. Each carbon nanotube film includes a number of successive and oriented carbon nanotubes joined end to end by van der Waals attractive force. When the carbon nanotube films are stacked sequentially, the orientations of the carbon nanotubes in two adjacent carbon nanotube films can be perpendicularly intersected.

One embodiment of a method for making a carbon nanotube film of the carbon nanotube structure 32 is described as the above steps S111 and S112. The carbon nanotube film of the carbon nanotube structure 32 also can be formed by entangled carbon nanotubes. Alternatively, the carbon nanotube film of the carbon nanotube structure 32 can be formed of a plurality of carbon nanotubes arranged isotropically.

In the step 220, the concentration of the protein solution 33 is in a range from about 0.01% (v/v %) to about 50% (v/v %). Preferably, the concentration of the protein solution 33 is in a range from about 0.1% (v/v %) to about 10% (v/v %). The solutes dissolved in the solution can be chosen from bovine serum, porcine serum, equine serum, goat serum or combination thereof. In one embodiment, the protein solution 33 is bovine serum solution with the concentration of about 2% (v/v %).

In the step 230, the protein solution 33 can be dropped on the carbon nanotube structure 32 by the steps of:

(231), fixing the carbon nanotube structure 32 at a frame 36, some portion of the carbon nanotube structure 32 being suspend from the frame 36;

(232), spreading the protein solution 33 on the carbon nanotube structure 32 by ejection, spray, or spin-coating; and

(233), removing the frame 36 to form the hydrophilic composite 30.

In the step 231, two opposite sides of the carbon nanotube structure 32 are exposed to surrounding environment. The frame 36 can be made of metal or wood. In one embodiment, the frame 36 is made of metal.

Accordingly, the present disclosure is capable of providing a hydrophilic composite. The hydrophilic composite including carbon nanotubes has the following benefits. First, the protein layer having soluble proteins covers at least one surface of the carbon nanotube structure such that the hydrophilic composite has good hydrophile for using in carious fields. Second, the carbon nanotube structure is capable of bending and employing to needed structures with different shapes such that the hydrophilic composite can be employed in biotechnology field. Third, the soluble proteins of the protein layer can also penetrate into the carbon nanotube structure through holes formed by mesh-like carbon nanotube structure such that the hydrophilic composite has array pattern, and thus the pure characteristic of the hydrophilic composite is improved.

It is to be understood that the above-described embodiments are intended to illustrate rather than limit the disclosure. Any elements described in accordance with any embodiments is understood that they can be used in addition or substituted in other embodiments. Embodiments can also be used together. Variations may be made to the embodiments without departing from the spirit of the disclosure. The above-described embodiments illustrate the scope of the disclosure but do not restrict the scope of the disclosure.

Depending on the embodiment, certain of the steps of methods described may be removed, others may be added, and the sequence of steps may be altered. It is also to be understood that the description and the claims drawn to a method may include some indication in reference to certain steps. However, the indication used is only to be viewed for identification purposes and not as a suggestion as to an order for the steps.

What is claimed is:

1. A hydrophilic composite, comprising:
a carbon nanotube structure having at least one carbon nanotube film; and
a protein layer covering at least a portion of one surface of the carbon nanotube structure, wherein the protein layer is coupled to the at least one carbon nanotube film.

2. The hydrophilic composite of claim 1, further comprising a substrate, wherein the carbon nanotube structure is disposed on the substrate.

3. The hydrophilic composite of claim 2, wherein the substrate is a hydrophobic substrate.

4. The hydrophilic composite of claim 2, wherein the substrate is a flexible substrate.

5. The hydrophilic composite of claim 2, wherein the substrate is silicon dioxide, glass, or ceramic.

6. The hydrophilic composite of claim 1, wherein the at least one carbon nanotube film is formed comprising a plurality of carbon nanotubes arranged substantially parallel to the surface of the carbon nanotube structure.

7. The hydrophilic composite of claim 6, wherein adjacent carbon nanotubes of the plurality of carbon nanotubes are combined and attracted to each other only by van der Waals attractive force therebetween.

8. The hydrophilic composite of claim 1, wherein the at least one carbon nanotube film is formed comprising a plurality of carbon nanotubes entangled with each other, or the at least one carbon nanotube film is formed comprising a plurality of carbon nanotubes arranged isotropically.

9. The hydrophilic composite of claim 1, wherein the carbon nanotube structure comprises a plurality of carbon nanotube films stacked together.

10. The hydrophilic composite of claim 9, wherein each of the plurality of carbon nanotube films comprises a plurality of carbon nanotubes orientated in one direction, an angle between the orientations of carbon nanotubes in two adjacent carbon nanotube films of the plurality of carbon nanotube films is about 90 degrees.

11. The hydrophilic composite of claim 1, wherein the protein layer comprises soluble protein.

12. The hydrophilic composite of claim 11, wherein the soluble protein comprises serum protein.

13. The hydrophilic composite of claim 1, wherein the protein layer penetrates into the carbon nanotube structure.

14. The hydrophilic composite of claim 1, where the surface is a flat surface or a curved surface.

15. A culture medium, comprising:
a substrate;
a carbon nanotube structure having at least one carbon nanotube film disposed on the substrate; and
a protein layer covering at least a portion of one surface of the carbon nanotube structure, wherein the protein layer is coupled to the at least one carbon nanotube film.

16. The culture medium of claim 15, wherein the substrate is a flexible substrate.

17. The culture medium of claim 15, wherein the substrate is silicon dioxide, glass, or ceramic.

18. The culture medium of claim 15, wherein the carbon nanotube film is formed comprising a plurality of carbon nanotubes arranged substantially parallel to the surface of the carbon nanotube structure.

19. The culture medium of claim 15, wherein the carbon nanotube structure comprises a plurality of carbon nanotube films stacked together.

20. The culture medium of claim 15, wherein the protein layer comprises soluble protein.
